(19) United States
(12) Patent Application Publication
(10) Pub. No.: US 2008/0220440 A1
(43) Pub. Date: Sep. 11, 2008

(54) Waveguide sensors optimized for discrimination against non-specific binding

(76) Inventors: M Selim Unlu, Jamaica Plain, MA (US); Bennett B Goldberg, Newton, MA (US); Colin Worth, Newton, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/333,864

(22) PCT Filed: Apr. 13, 2001
(86) PCT No.: PCT/US01/12339

§ 371 Date: Jan. 24, 2003

Related U.S. Application Data
(60) Provisional application No. 60/220,543, filed on Jul. 25, 2000.

(51) Int. Cl. G01N 33/53; C12M 1/34

(52) U.S. Cl. 435/7.1; 435/287.2; 356/73.1

(57) ABSTRACT

A system for interferometrically detecting the present of bound material using a wave propagating waveguide and the influence on propagation time of bound material in the proximity of the waveguide. The waveguide layer thickness and the radiation wavelength λ are selected so that the effects on phase difference between the two radiations applied in the beam are minimal in the region directly adjacent the surface of the waveguide, so as to unmask the influence of the more distant bound materials.

### CROSS REFERENCE TO RELATED APPLICATION

This application claims priority to prior provisional application, Ser. No. 60/220,543, filed Jul. 25, 2000, incorporated herein by reference.

### FIELD AND BACKGROUND OF THE INVENTION

Thin film waveguide, interferometric detection of specific target materials such as bound bio-components and mirco-organisms such as bacteria is based on the fact that such materials are typically surface bound on the waveguide. The desired bio or other material is often at a distance from the waveguide surface and are thus of reduced effect on the waveguide traveling wave and detected interferometrically with less sensitivity than materials, including environmental litter, debris and other contaminants etc. that are bound to the waveguide surface closer and whose influence on the traveling wave is more strongly felt in the interferometric analysis, masking the target material. In other cases the target specimen may be large relative to the litter and though being at the surface it still cannot be separately detected and distinguished from the litter. The effect of bound specimens or other materials is the result of their effect on the apparent index of refraction since it is the change in the index between the guiding layer and the bordering layers. As a result of this effect, the desired specimen may not be detectable, or separately detectable, being masked by the more strongly felt and closely bound litter.

While thin film technology is very effective in creating low cost structures, it is thus often unsuitable and it becomes necessary to use more expensive, slower and more sophisticated testing procedures for these purposes. This often means that testing for important bio-contaminants is unavailable due to cost considerations.

On the other hand, if a low cost and fast testing system were available, even of less precision that the more costly approaches, initial screening could be accomplished on a large number of cases at low cost, saving the higher cost refining testing to only those cases where there is a positive result from the economical test.

### SUMMARY OF THE INVENTION

The present invention solves this problem by providing at low cost a system for the thin film waveguide, interferometric detection of specific target materials such as bound bio-components and mirco-organisms such as bacteria. Such materials are typically either: bound on the waveguide at a distance from the waveguide surface and are thus of reduced effect on the waveguide traveling wave and detected interferometrically with less sensitivity than materials; or are large and if closely bound are detectable but not distinguishable from litter closely bound. The present invention reduces the influence of litter materials that are closely bound to the waveguide surface and whose influence on the traveling wave is more strongly felt or indistinguishable in the interferometric analysis. This is achieved by applying two radiations of different TE and TM modes and tuning the waveguide dimensions and light wavelength to a point where the difference in the changes of phase of each is a minimum for effects close to the waveguide surface but still of a significant change for those in or partly in the range of distances where materials of interest are bound.

A wave guiding layer of a dielectric material is provided of a predetermined thickness. The layer is bordered by layers of different index of refraction so as to confine a propagating wave, typically the lowest order light wave modes, TE₀ and TM₀ modes at for example 1300 nm. The two components of the beam are processed to interfere with each other, such as by separating and then recombining them to cause an interference pattern, the position of which can be measured to indicate the degree of phase shift of one relative to the other and in turn of the amount of material in the biolayer.

According to the present invention the waveguide layer thickness and the radiation wavelength λ are selected so that the effects on phase difference between the two radiations applied in the beam are minimal in the response to material in the region directly adjacent the surface of the waveguide, so as to unmask the influence of the more distant material.

### DESCRIPTION OF THE DRAWING

These and other features of the invention are more fully set forth below in the description of the invention and in the accompanying drawing of which:

FIG. 1A is a cross sectional view of a thin film waveguide for detecting surface target material shown along the direction of light wave propagation;

FIG. 1B is a perspective diagrammatic view of the waveguide channel in a waveguide of FIG. 1;

FIG. 2 is a sensitivity plot against waveguide thickness useful in understanding the invention;

FIGS. 3A and 3B are plots of differential sensitivity against distance from the waveguide useful in understanding the invention;

FIGS. 4 and 5 are radiation intensity diagrams for TE and TM modes of radiation in the waveguide and adjacent substrate and detection areas;

FIG. 6 is a diagrammatic view of system components for practicing the invention;

FIG. 7 is an exemplary view of a waveguide with fluid application elements and light injection and extraction gratings useful in the invention;

FIG. 8 is a plot illustrating the change in phase difference with specimens and buffer solutions at the waveguide surface useful in understanding the invention;

FIG. 9 is a diagram of an exemplary means for generating an interference pattern useful in understanding the invention; and

FIG. 10 illustrates an alternative waveguide shape.

### DETAILED DESCRIPTION OF THE INVENTION

The present invention contemplates a system for the thin film waveguide, interferometric detection of specific target materials such as bound bio-components and mirco-organisms such as bacteria by unmasking it from litter bound close to the waveguide surface. Target materials are in one case surface bound on the waveguide at a distance from the waveguide surface and are thus of reduced effect on the waveguide traveling wave and detected interferometrically with less sensitivity than non target material, including environmental litter, etc. that is bound to the waveguide surface closer and whose influence on the traveling wave is more strongly felt in the interferometric analysis. In another case, the target material is large and even if closely bound to the waveguide surface is indistinguishable from the closely bound litter. The effect on interferometric detection instrumentation of bound specimens or other materials is the result of their effect on the apparent index of refraction since it is the change in the index between the guiding layer and the bordering layers. Thus prior art systems typically are unable to detect objects at a distance in the presence of closely bound litter and are further unable to distinguish litter from large objects even if closely bound.

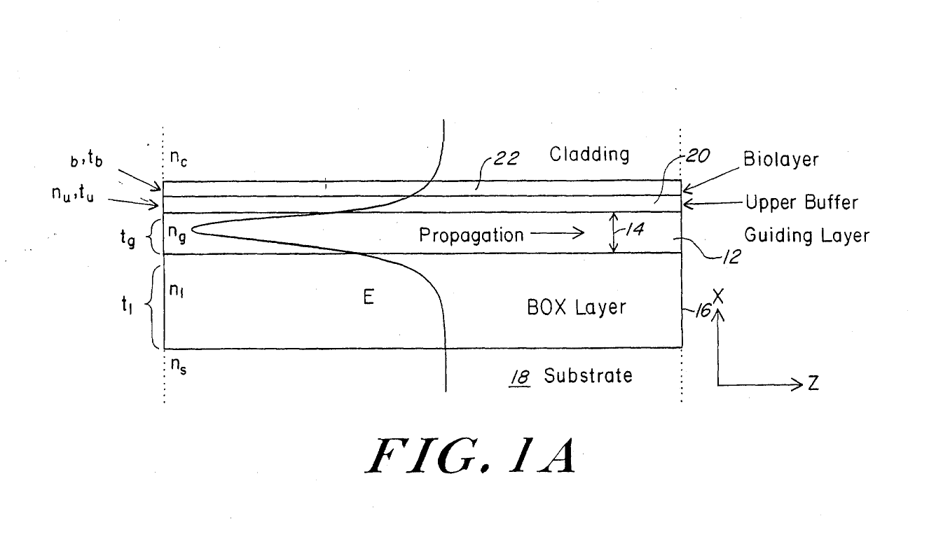

FIGS. 1A and 1B present a typical waveguide assembly for use in interferometric detection according to the invention. As show there, a dielectric layer 12 is provided of a predetermined thickness 14 and sufficiently wide in the direction in and out of the page to be of infinite width for analysis purposes. The layer 12 typically has a lower buffer layer 16 which in turn is formed on a substrate 18. Above the layer 12 is a further buffer layer 26 and above it a region 22 where distant target material 28a and large but closely bound target material 28b as well as non target materials 30 respectively are bound. Typically region 22 will be in a fluid as explained below.

The dielectric layer 12, commonly of silicon, doped glass or silicon-oxynitrides, has an index of refraction that differs from the layers that border it so as to confine a propagating wave 24 of wavelength λ, typically the lowest order light wave modes, TE₀ and TM₀ modes, with the electric field vectors respectively horizontal and vertical (nominally) in the view of the figures. The horizontal direction can be large but typically is sufficient to behave as though infinite. The effect of bound materials in the biolayer 22 is to influence the index of refraction which in turn effectively delays one or both modes, creating a phase shift in it or them at the output radiation 26. The two components of the beam 26 are then processed to interfere with each other, such as by separating and then recombining them to cause an interference pattern, the position of which can be measured to indicate the degree of phase shift of one relative to the other and in turn of the amount of material in the biolayer 22.

The closer, non target materials 30, which are typically unavoidable debris and contaminants by being closer will have a stronger influence on that process relative to the specific, distant, target materials 28a. In the case of large, closely bound particles 28b, detection of a phase shift is not conclusive of their presence since the phase shift could be attributable to closely bound litter instead.

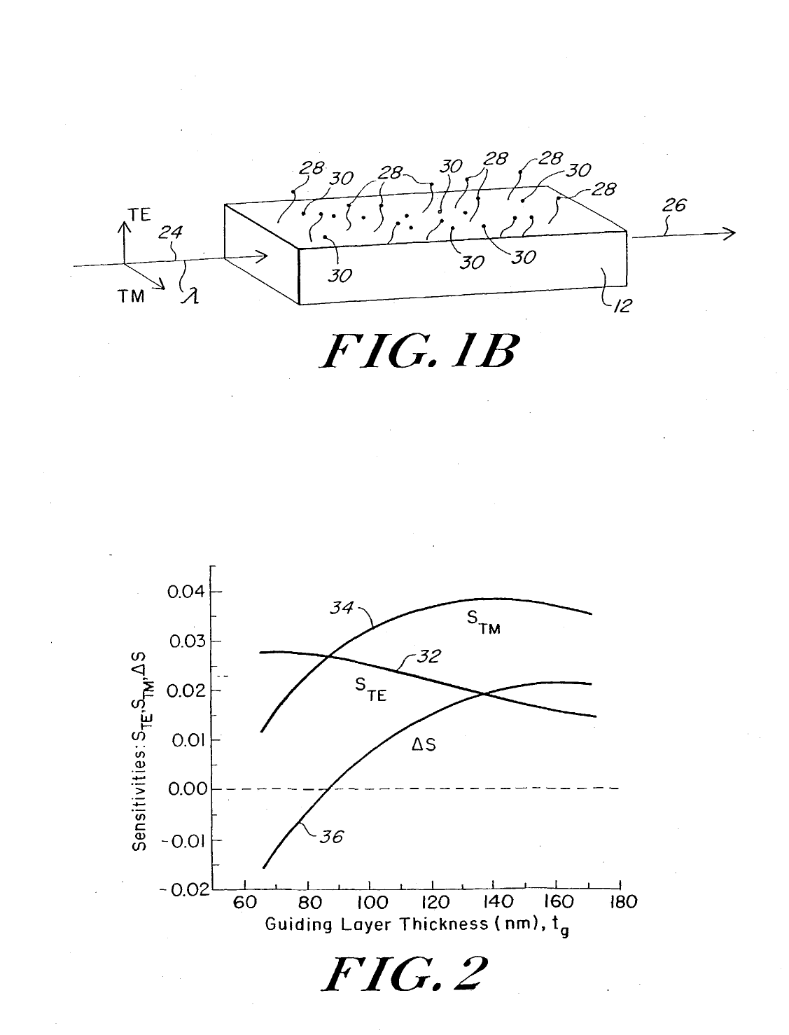

According to the present invention the guiding layer thickness 14 and the wavelength λ are selected so that the effects on phase difference between the two radiations applied in the beam 24 are minimal in the region directly adjacent the surface of the waveguide, layer 20, so as to unmask the influence of the more distant materials 28a or large materials 28b with distant components. FIGS. 2-5 show how this can be realized. In FIG. 1 the interferometric sensitivities of the system of FIGS. 1A and 1B are plotted for each of the TE₀ and TM₀ modes in curves 32 and 34. The difference between the curves is plotted as curve 36. As can be seen, the difference passes through a region of minimum value or zero at a specific thickness 14, in this case about 85 nm. This set of curves is also a function of the guiding material and wavelength λ. In a typical case the wavelength is about 632.8 nm from a HeNe laser and the construction is thin film construction of silicon nitride and silicon dioxide with the guiding layer having an index of refraction of 2.02 and the bordering or cladding layers with an index of 1.46. The modes are those given above and while other pairs of radiations may be used it is strongly preferred that only zero order modes be used.

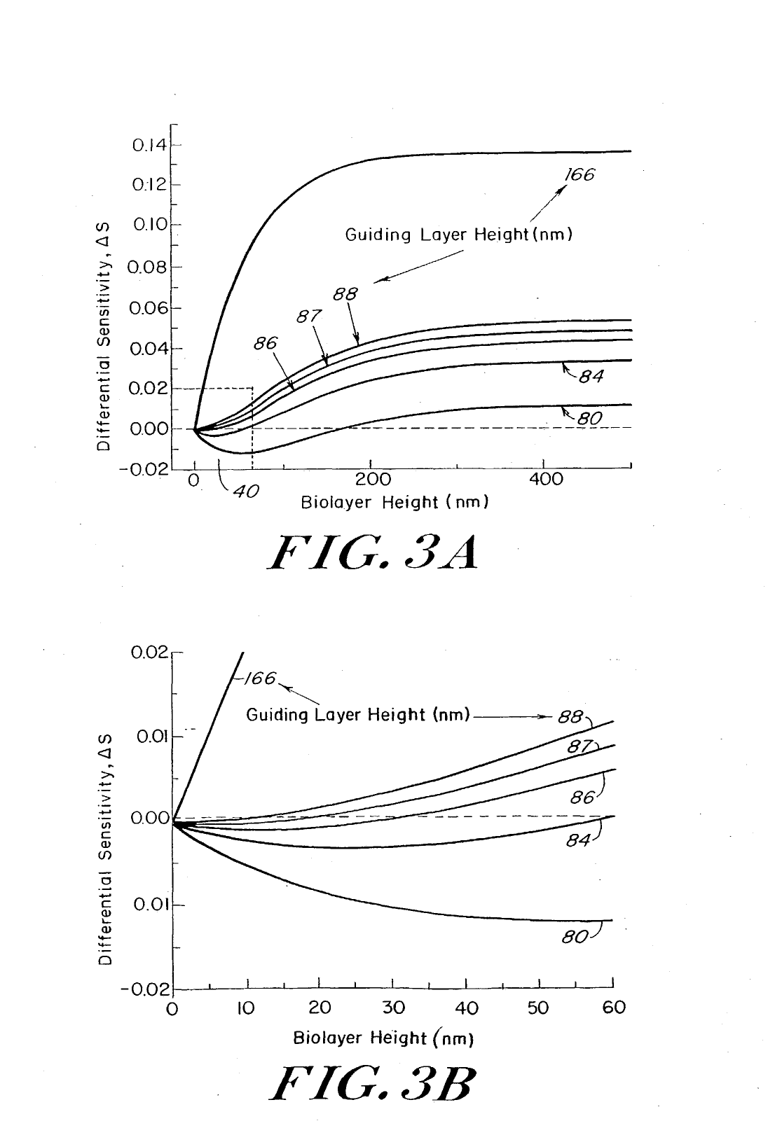

The interplay of thickness 14 and the difference in the phase change for the two radiation components is illustrated in a set of curves. FIG. 3B shows an amplified portion 40 of the FIG. 3A region where the invention is operative. As seen there, by changing the thickness 14 over a range of thickness from 80 to 88 nm, the minimum in sensitivity of the system to non specific material can be changed over a range of biolayer thickness from 10 to nearly 200 nm. Thus by appropriately tuning the wavelength and thickness, a sensor is realized that discriminates against material closer to the waveguide surface in the biolayer.

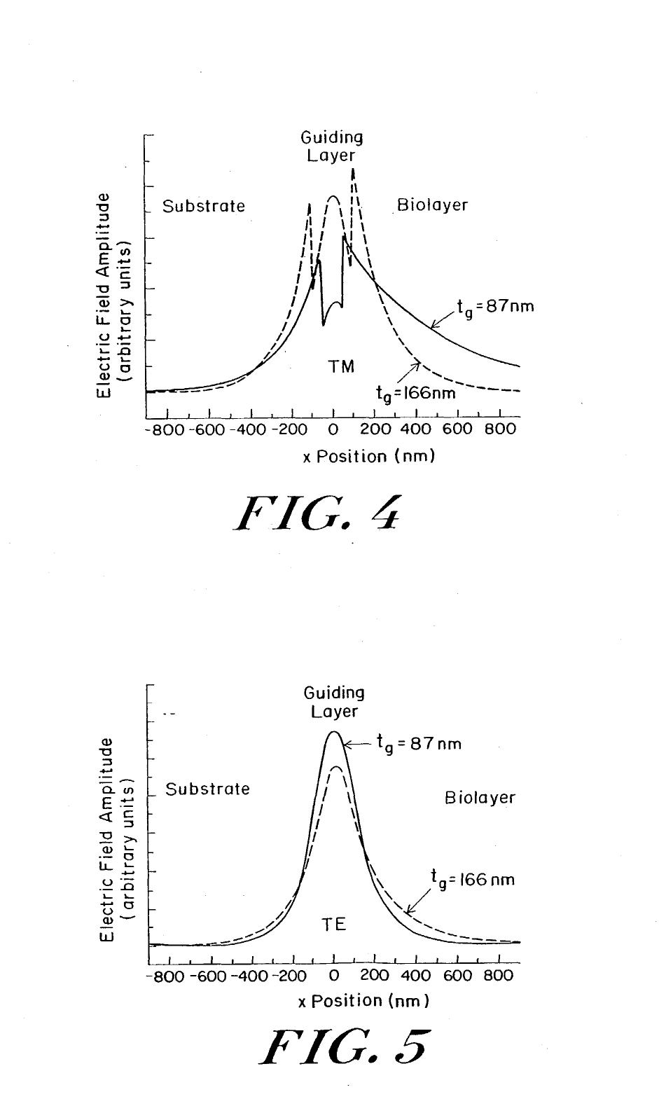

Seen from yet another perspective in FIGS. 4 and 5 is the influence on the amplitudes of the TE₀ and TM₀ modes in the assembly of guiding layer 12 and bordering layers, thereby indicating how the effect of the layer interfaces and material in the biolayer 22 affect the propagation time for the two modes at different guiding layer 12 thicknesses of 87 and 166 nm. As shown there, the electric field vector for the TM modes experience an abrupt discontinuity at the guiding layer 12 borders which is absent from the vector for the TE wave.

FIG. 6 illustrates more completely a system in which the waveguide layer 12 receives an input beam 24 and has an output beam 26. The output beam 26 is applied to beam separator 50, such as a polarization separator, which redirects the separate beams into converging or interfering beams 52 and 54. The converging beams 52 and 54 create an interference pattern, for example on the surface of a video detector 56 which can be applied through a processor 58 to display on a monitor 60 the interference pattern 62. The processor and/or the display 60 can identify by reference data any shift in the interference pattern indicative of the presence of material in the range of sensitivity as described above, and with greatly reduced effect from the closely bound material of non interest. FIG. 9 illustrates one realization of the interference optical assembly in which the beam 26 is split and then separate beams are converged by a prism 82, with an internal interface 83 onto a single detector 84, in a plane, which can include a video sensitive layer as described above.

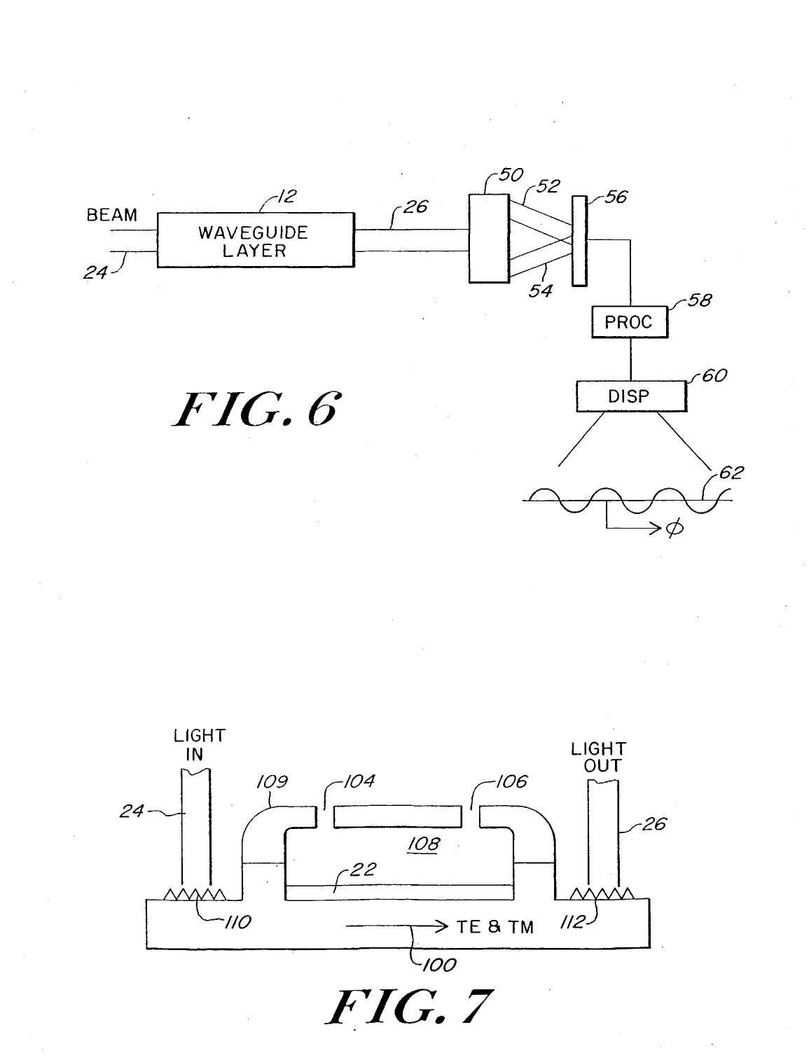

In actual practice of the invention it is convenient to flow the material to be analyzed over the surface of the waveguide in a fluid. For this purpose, the assembly of FIG. 7 is an example of how this is accomplished. As shown there the waveguide is fabricated as a waveguide assembly 100, typically as described above and has a fluid channel 102 formed above the surface surrounding the biolayer 22. Fluid is applied via inlet 104 and outlet 106. The immediate surface between the biolayer 22 and the waveguide is activated as is known in the art to bind to a desired material or organism in the fluid flowing through the channel 108 defined between inlet and outlet. The radiation in input beam 24 and output beam 26 can be applied and extracted through gratings 110 and 112 at opposite ends of the waveguide.

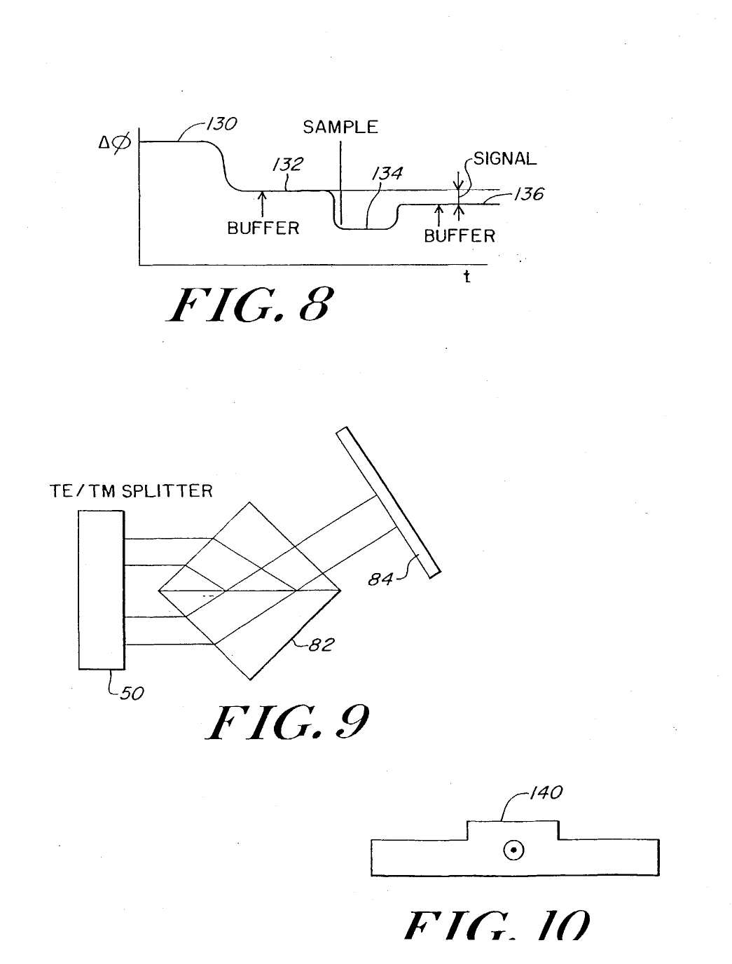

The fluid in channel 108 is typically applied as a sequence of fluids as illustrated in FIG. 8. Initially a buffer fluid as is known in the art is applied that changes the sensed phase difference to change from an ambient level 130 to a level 132 representing the presence of the buffer. Subsequently, a fluid of buffer and specimen is applied creating a further change to a level 134. Once there has passed sufficient time for the desired material, if present, to bind, a further and typically the same, buffer fluid is applied to purge the channel 108 of all unbound material. The system senses a new level 136. The difference between the levels 132 and 136 is the actual value sought as an indication of the presence of a material being tested for and in what quantity. If a positive result is obtained, testers will have achieved a high reliability fast result that can then be supplemented as desired with a more refined, costly and time consuming test.

FIG. 10 illustrates a modification to the guiding layer 12 by placing a ridge 140 in a central wave propagating region that in many cases will increase the sensitivity of the system. The ridge 140 produces a lateral confinement that can also support a single mode laterally and vertically.

The invention shown above is limited in scope only in accordance with the following claims.

1. A method for detecting a target material located within a first range of distances, above the surface of a waveguide of predetermined dimensions, and in the presence of a non target material in a second range of distances close to the waveguide surface, said first and second ranges being sufficiently close for material to effect therewithin propagation of radiation in said waveguide, said method comprising the steps of:
directing a first radiation of a selected wavelength and phase through said waveguide;
directing a second radiation of a selected wavelength and phase through said waveguide;
interferometrically detecting a phase difference between the first radiation and the second radiation as the first radiation and the second radiation are directed through said waveguide;
the detected phase difference having a relatively low first component effect provided by the selected wavelength of the first radiation and the selected wavelength of the second radiation cooperating with said predetermined dimensions of the waveguide to act on the phase difference caused by the non target material;
the detected phase difference having a relatively large second component effect provided by the selected wavelength of the first radiation and the selected wavelength of the second radiation cooperating with said predetermined dimensions of the waveguide to act on the phase difference caused by the target material; and
detecting the target material by comparing the first component effect and the second component effect.

2. The method of claim 1 further including the step of sensitizing said waveguide surface to bind said target materials.

3. The method of claim 2 wherein said sensitizing step includes sensitizing said surface to bio-materials which bind to the sensitized surface with at least a portion of said biomaterials within said first range of distances.

4. The method of claim 1 further including the step of directing a fluid containing said target materials across the surface of the waveguide.

5. The method of claim 4 wherein said fluid directing step includes the step of applying in sequence as a fluid across the waveguide surface a first applied buffer fluid, the target material containing fluid, and a second applied buffer fluid.

6. The method of claim 5 wherein said step of detecting a phase difference includes the step of detecting a phase difference shift between conditions of the first and second applied buffer fluids.

7. The method of claim 6 wherein said first and second applied buffer fluids are the same.

8. The method of claim 1 further including the step of applying the directed first and second radiations through gratings into said waveguide.

9. The method of claim 1 further including the step of forming said waveguide from a thin film dielectric material.

10. The method of claim 9 wherein said dielectric material is silicon, doped glass, or silicon-oxynitride.

11. The method of claim 10 wherein said waveguide is at least partially bordered other than at said surface by layers with substantially different indices of refraction thereby to substantially confine the directed first and second radiation to said waveguide.

12. The method of claim 10 wherein said waveguide is formed of silicon nitride silicon dioxide and has a thickness of at least 0.08 nm.

13. The method of claim 12 wherein said first and second radiations are the TE₀ and TM₀ modes.

14. The method of claim 1 wherein said detecting step further includes forming an interference pattern between the first and second radiations exiting said waveguide.

15. The method of claim 14 further including the step of detecting the phase difference as a shift in the interference pattern against a standard condition of substantially no bound target material.

16. The method of claim 1 wherein said directing step includes the step of directing said first and second radiations to a waveguide having a ridged cross section.

17. The method of claim 1 wherein said directing step includes the step of applying said first and second radiations in a beam of smaller cross section dimensions than that of the cross section dimensions of said waveguide.

18. The method of claim 1 further including the step of forming said first and second radiations by separating the radiation in a laser beam into different modes.

19. The method of claim 18 wherein said different modes are the TE₀ and TM₀ modes.

20. Apparatus for use in the method of claim 1 including:
a thin film waveguide having a transverse wave supporting channel;
a surface bordering said waveguide for exposure to target material;
said surface partially transmissive to transverse waves and thereby causing a phase sensitive delay in the transmission of said transverse waves in said waveguide as a function of target material beyond said surface and not immediately adjacent to said surface.

21. The apparatus of claim 20 further including a fluid path adjacent said surface for applying material into the vicinity of said surface to thereby effect a delay in the propagation of said transverse waves to a degree representative of the target material.

22. The apparatus of claim 20 further including a sensitized layer on said waveguide surface to bind said target materials.

23. The apparatus of claim 20 wherein said surface is sensitized to bio-materials which bind to the sensitized surface within said first range of distances.

24. The apparatus of claim 20 further including means for directing a fluid containing said target materials across the surface of the waveguide.

25. The apparatus of claim 24 wherein said fluid directing means includes means for applying in sequence as a fluid across the waveguide surface a first applied buffer fluid, the target material containing fluid, and a second applied buffer fluid.

26. The apparatus of claim 25 further including means detecting a phase difference shift between conditions of the application of the first and second applied buffer fluids.

27. The apparatus of claim 20 wherein said first and second applied buffer fluids are the same.

28. The apparatus of claim 20 further including gratings for applying the directed first and second radiations into said waveguide.

29. The apparatus of claim 20 wherein said waveguide is a thin film dielectric material.

30. The apparatus of claim 29 wherein said dielectric material is silicon, doped glass or silicon-oxynitride.

31. The apparatus of claim 30 wherein said waveguide is at least partially bordered other than at said surface by layers with substantially different indices of refraction thereby to substantially confine the directed first and second radiation to said waveguide.

32. The apparatus of claim 30 wherein said waveguide material is silicon nitride silicon dioxide and has a thickness of at least 0.08 nm.

33. The apparatus of claim 32 wherein said first and second radiations are the TE₀ and TM₀ modes.

34. The apparatus of claim 20 further including means for forming an interference pattern between the first and second radiations exiting said waveguide.

35. The apparatus of claim 34 further including means for detecting a phase difference representative of the presence of a target material as a shift in the interference pattern against a standard condition of substantially no bound target material.

36. The apparatus of claim 20 wherein said waveguide has a ridged cross section.

37. The apparatus of claims 20 wherein said first and second radiations are in a beam of smaller cross section dimensions that the cross section dimensions of said waveguide.

38. The apparatus of claim 20 further including means for separating the radiation In a laser beam into different modes.

39. The apparatus of claim 38 wherein said different modes are the TE₀ and TM₀ modes.

40. The method of claim 1 wherein said target materials are bio specimens.

41. The apparatus of claim 20 wherein said target materials are bio specimens.

42. A method for detecting a presence of a target material at a distance from a surface of a waveguide in a presence of a non target material closer to said surface comprising the steps of:
selecting a wavelength of a radiation;
selecting a thickness of said waveguide;
having the non target material bound to said surface more closely than a target material bound to said surface;
applying the radiation in a TE₀ mode to said waveguide;
applying the radiation in a TE_{M} mode to said waveguide;
combining the radiation in the TE₀ mode and the radiation in a TE_{M} mode to create an interference pattern;
the waveguide thickness being selected to enhance an effect of target material on said interference pattern relative to an effect of non target material; and
detecting the presence of the target material based on the interference pattern.

43. (canceled)

44. The method of claim 42 wherein the step of detecting includes a step of detecting the presence of the non target material based on the interference pattern.
